(19) United States
(12) Patent Application Publication
(10) Pub. No.: US 2011/0203963 A1
(43) Pub. Date: Aug. 25, 2011

(54) Transdermal alcohol characterized toiletry

(76) Inventor: PAMELA K. TANNER, GOLDEN, CO (US)

(21) Appl. No.: 12/931,423

(22) Filed: Feb. 2, 2011

Related U.S. Application Data
(60) Provisional application No. 61/338,808, filed on Feb. 25, 2010.

(51) Int. Cl. B65D 85/84; A61K 8/00; A61Q 15/00; A61Q 5/02

(52) U.S. Cl. 206/524.1; 424/65

(57) ABSTRACT

A toiletry product, adapted for use by an individual in a transdermal alcohol monitoring program, is provided. The product is formulated as toiletry composition which is substantially alcohol-free. The product is characterized to determine that it does not cause a positive interferant result when sampled in proximity to a transdermal alcohol content monitoring device. The product is packaged and identified as a product approved for use in an alcohol monitoring program when orally or topically applied.

### CROSS REFERENCE TO RELATED APPLICATIONS

Pursuant to 35 U.S.C. 119(e), applicant claims the benefit of U.S. Ser. No. 61/338,808 filed, pursuant to 35 U.S.C. 111(b), on Feb. 25, 2010.

### STATEMENT OF FEDERALLY SPONSORED RESEARCH

Not Applicable.

### BACKGROUND OF THE INVENTION

1. Field of the Invention

The present invention relates to transdermal alcohol monitoring. In particular, it relates to a non-interferant toiletry for use by one enrolled in a transdermal alcohol content monitoring program.

2. Description of the Related Art

Courts and treatment programs often require the abstinence from alcohol as a condition of pretrial supervision, sentencing, or program completion. One such method of monitoring compliance with the abstinence condition is a court ordered requirement that the individual wear a tamper proof transdermal alcohol monitoring device. Other such devices include an order requiring the use of a transdermal alcohol monitoring device coupled to an ignition interlock device for operating a motor vehicle.

Transdermal alcohol monitors are securely attached of the human subject to be monitored. As alcohol is ingested orally, it is absorbed into the body's blood and distributed throughout the body via the circulatory system. Alcohol is eliminated from the body either by metabolism or excretion. Alcohol is excreted unchanged whenever water is removed from the body, breath, urine, perspiration and saliva, which permits an accurate measurement of alcohol concentration in the body by way of perspiration. This perspiration can be used to obtain a transdermal measurement of blood alcohol concentration, which is referred to herein as the Transdermal Alcohol Concentration (“TAC”).

Blood alcohol monitors are know which differentiate between a positive reading, caused by an external interferant source of ethanol, and a positive reading caused by the consumption of alcohol. One such device is disclosed in U.S. Pat. No. 5,220,919 to Phillips et al. The device measures ethanol levels expelled through a subjects' skin. The device differentiates between a positive reading caused by an external, interferant, source of ethanol and a positive reading caused by consumption of alcohol The device is attached to the subject by a conductive strap, monitors the skin temperature of the subject prior to performing the alcohol test, and measures the distance from the subjects skin. A gas content sensor uses an electro-chemical fuel cell with a sample port and an ethanol selective, non-hydrophilic, membrane to measure the ethanol content in the perspiration. An algorithm is used to make a comparison of absorption rates and burn off rates as a function of time in order to determine if a positive reading is due to an interferant.

Another such device is disclosed in U.S. Pat. No. 7,641,611 to Hawthorne, et. al. This device includes a device having analog and digital sides which are typically attached to the ankle of the individual to be monitored. The analog side includes a sampling circuit which draws a measured insensible skin perspiration sample from the skin of the subject. The perspiration sample is measured with an electrochemical fuel cell. A distance measurement of the device from the skin of the subject and temperature of the sample are also monitored along with the transdermal alcohol content, and converted to digital signals which are output for upload to a central monitoring station.

Toiletry products used in washing or caring for the appearance of an individual such as shampoo, deodorant, and soaps, often contain ethanol as a solvent. When the ethanol is absorbed through the skin it can interfere with a TAC measurement causing a false positive result. The false positive result is referred to as an interferant. The interferant result may be defined as any reading indicating a positive TAC, but where the positive TAC is caused by some sort of topical ethanol application to the skin or spilled/poured onto the Monitoring Device, but one which is not the result of ingesting alcohol into the body by drinking.

While the foregoing devices for blood alcohol monitoring include differentiation mechanisms which are useful in the monitoring of an individual's sobriety it is clear that a false positive TAC result is potentially devastating to the individual to be supervised. This false positive result subjects the individual to further hearings on the matter with the court and possibility of causing a revocation of probation or further loss of driving privileges. Moreover, individuals to be monitored are typically advised of this potentially devastating affect and warned at the onset to use only those toiletries which do not contain alcohol. However, a problem exists in that the subject is then required to undergo an immediate and extensive search for products which would not subject the individual to a false positive TAC result. This problem is further aggravated by the fact that, unless the individual has an understanding of alcohol chemistry, such as the difference between isopropyl alcohol and ethanol, there is no guarantee that the toiletry, purchased as a non-alcohol containing composition, will not indeed cause a false positive result when its metabolites are tested in proximity to an electrochemical fuel cell. Thus, what is needed is a readily available toiletry, or kit, for use by an individual under supervision which will not cause a false TAC monitoring result. The present invention satisfies this need.

### BRIEF SUMMARY OF THE INVENTION

It is therefore an object of the present invention to provide a toiletry which does not cause a false positive TAC result when applied to the skin.

It is another object of the present invention to provide an approved toiletry which is easily identified for purchase as one which does not cause a false positive TAC result when applied to the skin.

To overcome the problems of the prior art methods and in accordance with the purpose of the invention, as embodied and broadly described herein, briefly a toiletry product, adapted for use by an individual in a transdermal alcohol monitoring program, is made by a process. The product is formulated as toiletry composition which is substantially alcohol-free, and characterized to determine that it does not cause a positive interferant result when sampled in proximity to a transdermal alcohol content monitoring device. The product is packaged and identified as a product approved for use in an alcohol monitoring program.

Additional advantages of the present invention will be set forth in part in the description that follows and in part will be obvious from that description or can be learned from practice of the invention. The advantages of the invention can be realized and obtained by the apparatus particularly pointed out in the appended claims.

### BRIEF DESCRIPTION OF THE SEVERAL VIEWS OF THE DRAWINGS

The accompanying drawings, which are incorporated in and which constitute a part of the specification illustrate at least one embodiment of the invention and, together with the description, explain the principles of the invention.

FIG. 1 is a flow chart of the process for making the toiletry product.

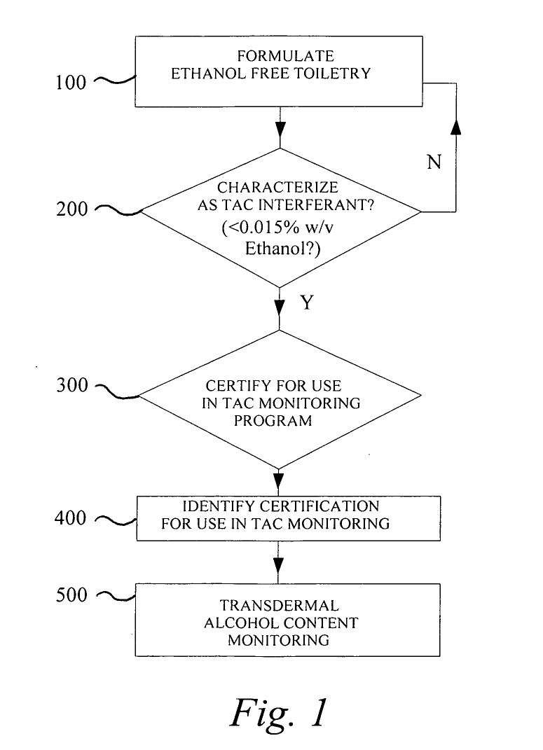

FIG. 2 is a perspective view a preferred packaging of the toiletry with certifying identification indicia and where the toiletries are a kit including a shampoo and bar soap.

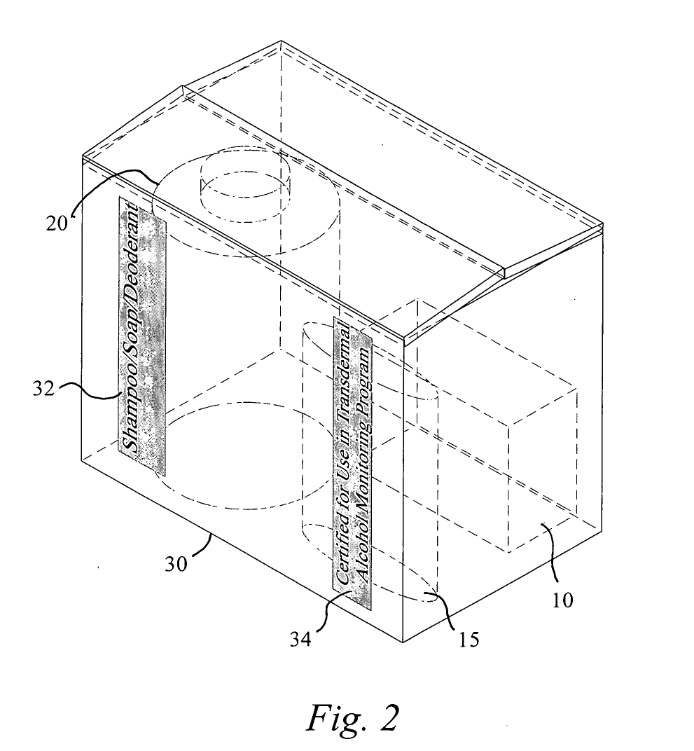

### DETAILED DESCRIPTION OF THE INVENTION

Unless specifically defined otherwise, all technical or scientific terms used herein have the same meaning as commonly understood by one of ordinary skill in the art to which this invention belongs. As used herein the term “TAC” means a transdermal alcohol concentration result measured using an electrochemical alcohol sensor. The term “interferant” means a sample which produces a positive TAC result, but which is not caused by consumption of alcohol.

Although any of the methods and materials similar or equivalent to those described herein can be used in the practice or testing of the present invention, the preferred methods and materials are now described. Reference will now be made in detail to the presently preferred embodiments of the invention, examples of which are illustrated in the accompanying drawings wherein like numerals represent like features of the invention.

The toiletry product 10, 20, in accordance with the present invention, is made by a process for ensuring that it does not produce a false positive TAC result. The toiletry is preferably formulated 100 as an alcohol-free composition for use in washing, cleaning or caring for the appearance of an individual, such as a shampoo, deodorant, or soap. The formulation may, but need not, include other compounds such as fragrances, conditioners, and colorings in any manner as is well known in the art. The formulation is characterized 200, by chemical analysis, so that a positive TAC result is not obtained when using the toiletry while enrolled in a transdermal alcohol content monitoring program. The toiletry 10, 20 is packaged and identified 30, 34 on the package 30 as an approved product for such use when applied topically to the skin.

The toiletry product 10, 20 is desirably formulated 100 in a wide variety of compositions for subsequent packaging as a kit 30 for purchase by an individual enrolled in an alcohol monitoring program. As such, the kit 30 would desirably include interferant-free antiperspirant gels 15, mouthwash, bar soap 10, shampoo 20 and/or fragrant body lotions and creams. Many formulations of alcohol-free toiletries have entered the market and are broadly disclosed. For example, alcohol-free transparent products have been made possible which are based on so-called microemulsions. These have great advantage that even relatively large amounts of various oils can stably be incorporated. Formulations of this type are in principle obtainable by means of phase-inversion temperature technology (PIT) or high-pressure homogenization. One such example includes alcohol-free, oil-in-water (O/W), type emulsions which include less that 20% by weight of one or more O/W emulsifiers selected from a group of polyethoxylated O/W emulsifiers, polypropoxylated O/W emulsifiers, and combinations thereof. Antiperspirants formulations are disclosed in U.S. Pat. No. 7,282,197 to Khiet, et al. Also, for example, disclosed in U.S. Pat. No. 5,817,295 to Chaudhari et al, is an alcohol free mouthwash formulation including a blend of essential oils, methyl salicylate, menthol and a tripartite blend of peppermint flavor oils that are dissolved in solution using a non-ionic surfactant. Another example is a bar soap which is formulated as monohydric alcohol-free, transparent, composition and disclosed in U.S. Pat. Ser. No. 6,462,002, to Saxena et al. This formulation includes from about 38% to about 96% of one or more humectant solvents and from about 3% to about 20% of insoluble and/or soluble fatty acids soaps, with at least 3% insoluble fatty acid soap. The weight ratio of humectant solvent to insoluble fatty acid soap ranges from about 3:1 to about 23:1. In yet another example, disclosed in U.S. Pat. No. 6,248,339, to Knitowski, is an alcohol-free fragrant body lotion or cream consisting of cyclomethicone/dimethicone cross-polymer gel, a fragrance, an opacifying agent, an isononyl isonanoate, and at least one hydrocarbon which includes at least one of isohexadecane and isododecane, and, if isohexadecane is present, at least one hydrogenated styrene copolymer. The foregoing formulation disclosures are incorporated by reference. Other such formulations include alvera all natural roll-on deodorants, Toms of Maine™ toothpaste, Pure Life™ chamomile products such as bar soap and conditioner, and Natures Gate™ moisturizing shampoo.

As one may well appreciate from the above, a wide variety of alcohol-free formulations may be used as precursors for characterization and approval for use with the present invention. Here, the toiletry is a product for washing or caring for the appearance of an individual. The toiletry is then characterized 200 by qualitative or quantitative chemical analysis for topical use of the individual while enrolled in a transdermal alcohol content monitoring program. Characterization analysis of the formulated composition is carried out using any method which is well known in that art for interferant and/or ethanol analysis, such as gas or liquid chromatography. The analytical methods desirably characterize the formulated sample to log a positive interferant result where the formulation sample is found to contain greater than 0.0155% weight/volume of ethanol. However, in a preferred embodiment of the present invention and to ensure that the formulated composition does not generate an interferant-positive result, characterization is desirably carried out using a gas content alcohol sensor, in situ, where the formulated composition is topically applied and the individual is subject to transdermal alcohol monitoring by any method sanctioned for use by a predetermined monitoring program. Such blood alcohol monitoring systems and baseline data are disclosed in U.S. Pat. No. 5,220,919 to Phillips, et al. which is incorporated by reference. In this manner, the sensor contains an electro-chemical fuel cell which contains an opening, for use as a sample port, to allow a volatized air sample, of the formulation or perspiration, to enter the fuel cell. In order to ensure that the sample port contains the proper gases to be analyzed, an ethanol selective, non-hydrophilic membrane is placed across the sample port of the fuel cell. This membrane improves the selectivity of the sensor to ethanol in the same manner as the transdermal alcohol content monitor used in supervising the individual. The output of the fuel cell is an electrical current which is proportional to the amount of alcohol present in the air within the sample port. The sample port is place at a predetermined distance, typically 1-5 millimeters, above the subject's skin. The sensor measures the amount of alcohol being emitted through the subject's skin in sample species including water, oxygen, carbon dioxide, and even alcohol. Such transdermal alcohol sensors useful in the practice or testing of the present invention are available from Giner, Inc., Newton, Mass. The sensor may be worn around the wrist, arm, leg, torso, or neck for characterization and monitoring of the formulated composition. In and even more preferred embodiment of the present invention, characterization 200 further includes the use of sensors which monitor the skin temperature of the subject prior to performing the characterization test, and measures the distance from the subjects skin during the characterization step.

Finally, the characterized formulation is approved for use, in by an individual enrolled in a transdermal alcohol monitoring program. Approval is desirably made by certification 300. In this manner, the product is transferred for packaging and identification 400 of the certified products for use. In the preferred embodiment, the toiletry is packaged as a kit 30, for use by the individual to be monitored. The kit 30, includes a combination of those toiletries, such as a bar soap 10, deodorant 15, and shampoo 20 which are typically occasioned for use by an individual in his/her personal care throughout a given day, week, or month. Approved use of the characterized toiletry is desirably identified by certification indicia 34 of the products 10, 15, and 20 included in the kit 30. In this manner, the kit 30 is readily available for purchase or use from either persons connected with supervising the monitoring program, or in an over-the-counter retail market through one's local drug store.

While the present invention has been described in connection with the embodiments as described and illustrated above, it will be appreciated and understood by one of ordinary skill in the art that modifications may be made in the toiletry, in accordance with the present invention, without departing from the true spirit and scope of the invention as described and claimed herein.

I claim:

1. A toiletry product adapted for use by an individual in a transdermal alcohol monitoring program made by a process, comprising the steps of:
(a) formulating a toiletry composition which is substantially alcohol-free; and
(b) characterizing the composition to determine that it does not cause a positive interferant result when sampled in proximity to a transdermal alcohol content monitoring device.

2. The toiletry product made by the process according to claim 1, wherein characterization is determined by chromatography.

3. The toiletry product made by the process according to claim 1, wherein characterization is determined by an electrochemistry sensor.

4. The toiletry product made by the process according to claim 1, wherein the alcohol and interferant are ethanol.

5. The toiletry product according to claim 1, wherein the transdermal alcohol content monitoring device includes a passive electrochemical fuel cell, a temperature sensor, and an infrared sensor.

6. The toiletry product according to the process of claim 1, further comprising the step of transferring the toiletry to a package having a transdermal alcohol content monitoring certification identification indicia.

7. The toiletry product according to the process of claim 3, wherein characterization is further determined with body temperature and proximity data.

8. The toiletry product according to claim 4, wherein the interferant concentration is less than 0.0155% sample weight/volume.

9. The toiletry product according to claim 4, wherein the ethanol concentration is less than 0.0155% sample weight/volume.

10. The toiletry product according to claim 6, wherein the package comprises a kit including at least two toiletry products.

11. The toiletry product according to claim 10, wherein the toiletry products are a shampoo and deodorant.
